(19) United States
(12) Patent Application Publication
(10) Pub. No.: US 2026/0273255 A1
(43) Pub. Date: Sep. 17, 2026

(54) Microneedles And Related Methods

(71) Applicant: THE TRUSTEEES OF COLUMBIA UNIVERSITY IN THE CITY OF NEW YORK, New York, NY (US)

(72) Inventors: Tongalp TEZEL, New York, NY (US); Ahmet HONDUR, New York, NY (US); Jeffrey W. KYSAR, New York, NY (US); Anil K. LALWANI, New York, NY (US); Dvir KOENIGSTEIN, New York, NY (US); Chaoqun ZHOU, New York, NY (US); Onur INAM, New York, NY (US)

(21) Appl. No.: 19/670,499

(22) Filed: May 7, 2026

Related U.S. Application Data
(63) Continuation-in-part of application No. PCT/US2024/055171, filed on Nov. 8, 2024.
(60) Provisional application No. 63/597,466, filed on Nov. 9, 2023.
(60) Provisional application No. 63/912,610, filed on Nov. 6, 2025.

(51) Int. Cl. A61M 37/00

(52) U.S. Cl. CPC A61M 37/0015; A61M 2037/003; A61M 2037/0061; A61M 2210/0612

(57) ABSTRACT

Provided herein at microneedles that include a base; one or more extensions, such as wings; a body; and a tip, which tip can, in some embodiments, be curved. The design allows the user to align the microneedle parallel with the blood vessel into which the microneedle is inserted, as the one or more extensions can stabilize the microneedle when inserted and also oppose excessively deep insertion of the microneedle. The disclosed design enables injections and aspirations to be made in a minimally damaging fashion, thereby enabling improved cannulation of blood vessels in the retina. The microneedles can be used, for example, to remove occlusions in these blood vessels by injecting an agent or by simply applying hydrodynamic forces, as well as selective treatment of intraocular tumors by microablation or infusion of chemotherapeutic agents.

### RELATED APPLICATIONS

The present application claims priority to and the benefit of U.S. patent application No. 63/912,610, “Microneedles And Related Methods For Retinal Intravascular And Optic Nerve Head Injections,” filed Nov. 6, 2025. The present application is also a continuation-in-part of international application no. PCT/US2024/055171, “Microneedles and Related Methods,” filed Nov. 8, 2024, which claims priority to and the benefit of U.S. patent application No. 63/597,466, “Microneedles And Related Methods,” filed Nov. 9, 2023. All foregoing applications are incorporated herein by reference in their entireties for any and all purposes.

### TECHNICAL FIELD

The present disclosure relates to the field of surgical instruments, in particular the field of ophthalmic instruments.

### BACKGROUND

Performing injections on small blood vessels or other vasculature poses unique challenges to medical providers, as existing surgical tools used to target small blood vessels (such as retinal veins and arterioles, for example) lack precision and can even cause excess trauma to the tissue in some instances. Accordingly, there is a long-felt need for improved surgical instruments useful in performing subretinal and other intravascular injections.

### SUMMARY

In meeting the described long-felt needs, the present disclosure provides microneedles that are useful in, for example, retinal endovascular surgery and other surgical procedures requiring high-precision minimally invasive cuts into delicate tissue and/or cannulation of the micro vessels. The disclosed technology also has applications in other delicate tissues-such as nervous tissue and microvasuclature- and can also be used for drug delivery to retinal tissues and extraction of subretinal or intraretinal fluid collections.

As described herein, a microneedle can include a base, wings, a body, and a tip, which tip can, in some embodiments, be curved. A microneedle can also include one or more lumens that allow for injection and/or aspiration. Aspiration or injection can take place sequentially, in any order. A microneedle can be used only for aspiration; a microneedle can also be used for injection. In some embodiments, aspiration and injection can take place simultaneously. A lumen can include a straight portion, a curved portion, or both. The disclosed design enables injections to be made behind the retina in a minimally damaging fashion, as well as allowing one to cannulate blood vessels in the retina. This technology can also be used, for example, to remove occlusions in these blood vessels by injecting an agent (TPA, as but one example) or by simply applying hydrodynamic forces, as well as selective treatment of intraocular tumors by microablation or infusion of chemotherapeutic agents.

The wings can be used to align with the vessel that will be cannulated; the wings allow a user to more accurately position and orient the needle during the procedure. Further, retinal surgeries commonly employ “portals,” which are short tubes that provide a safe route into the eye. To make the disclosed microneedle compatible with these portals, the wings of the disclosed microneedles can be collapsible. In this way, the microneedle can pass through the small portal and then open up once the microneedle is inside the eye, offering the surgeon the control they need.

A microneedle can, in some embodiments, be blade-like in configuration so as to allow for cutting into the tissue with more precision, thereby improving outcomes. Such a blade can include a serrated edge, and the cutting edge of a microneedle can, in some embodiments, be arranged perpendicular to the perforation direction so as to facilitate insertion.

In one aspect, the present disclosure provides a microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis; and a plurality of wings extending outwardly from the base, the plurality of wings being configured to contact a tissue into which the tip is inserted.

Also provided is a microneedle, comprising: a base disposed at a proximal end of the microneedle; a tip disposed at a distal end of the microneedle, the tip extending along a tip longitudinal axis, a shaft disposed between the base and the tip, at least one lumen defining a channel providing fluid communication within at least the shaft of the microneedle; an opening defined at the distal end of the lumen and at a loci on the shaft, wherein the opening is optionally offset from the tip longitudinal axis; and at least one extension extending from the base, the at least one extension configured to contact a tissue into which the tip is inserted, the at least one extension comprising at least one of a wing and a collar.

Further provided is a microneedle, comprising: a base disposed at a proximal end of the microneedle; a tip disposed at a distal end of the microneedle, the tip extending along a tip longitudinal axis, a shaft disposed between the base and the tip, wherein the shaft defines at least one edge extending distally toward and to the tip, the at least one edge being serrated; optionally, at least one lumen defining a channel providing fluid communication within at least the shaft of the microneedle; and optionally, an opening defined at the distal end of the lumen and at a loci on the shaft, wherein the opening is optionally offset from the tip longitudinal axis.

Additionally disclosed is a microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, wherein the shaft defines a profile that deviates from the shaft longitudinal axis, the shaft optionally defining at least one of a curved profile and an angled profile, and the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis.

Further disclosed is a microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis; and a plurality of wings extending outwardly from the base, the plurality of wings being configured to contact a tissue into which the tip is inserted, at least one of the plurality of wings lying perpendicular to a plane in which the shaft longitudinal axis and the tip longitudinal axis lie.

Also provided is a method, comprising delivering a fluid to a subject or withdrawing a fluid from a subject using one or more microneedles according to the present disclosure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1A provides a depiction of an exemplary microneedle according to the present disclosure.

FIG. 1B illustrates a microneedle according to the present disclosure approaching the retinal venule during an ex vivo experiment.

FIG. 1C illustrates balanced salt solution (BSS) injection into the vessel of FIG. 1B after having access to its lumen. The blood column in the retinal vessels moves as it is perfused.

FIG. 2 provides a depiction of an exemplary microneedle according to the present disclosure.

FIG. 3 provides a depiction of an exemplary microneedle according to the present disclosure.

FIG. 4 provides a depiction of a serrated microneedle shaft according to the present disclosure.

FIG. 5 provides a depiction of an exemplary microneedle according to the present disclosure, mounted on a medical instrument.

FIG. 6 provides a depiction of an exemplary microneedle according to the present disclosure.

FIG. 7 provides a depiction of an exemplary microneedle according to the present disclosure.

FIG. 8 provides a schematic of a microneedle mounted on a blunt needle and enclosed within a metal protective sheath for safe insertion through a trocar.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4.

Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

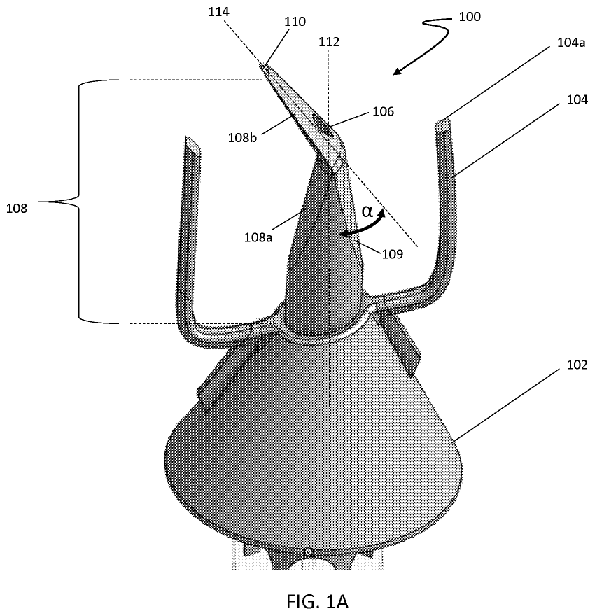

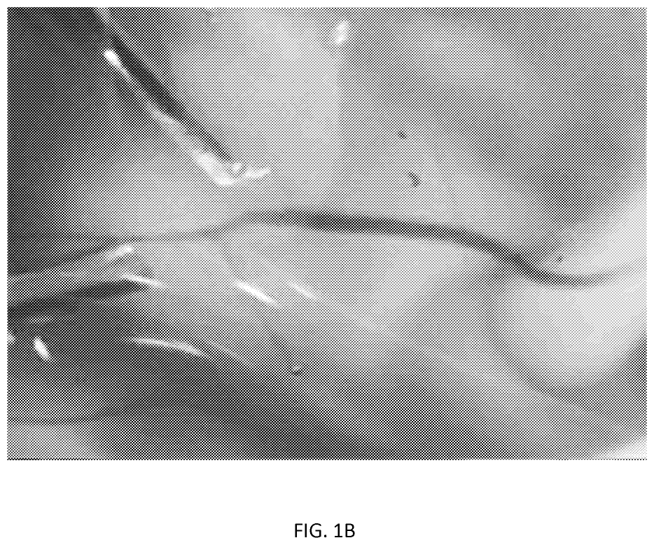

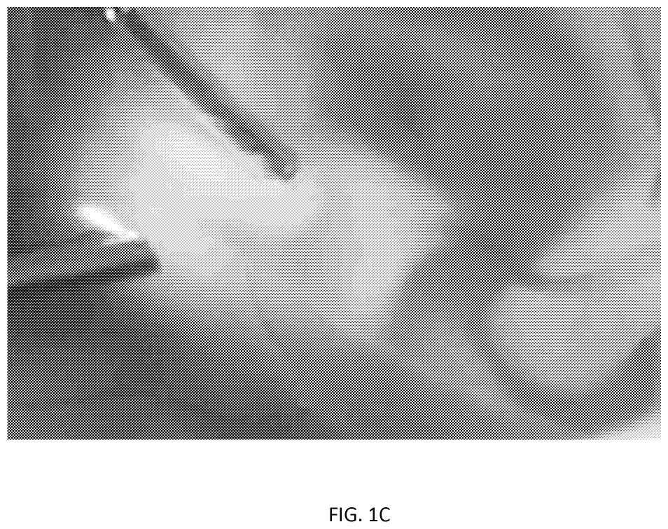

FIG. 1 provides a depiction of an exemplary microneedle 100 according to the present disclosure. As shown, microneedle 100 can include a base 102. Shaft 108 can extend distally from base 102; at least a portion of shaft 108 can extend along shaft longitudinal axis 112.

Shaft 108 can define a profile that deviates from shaft longitudinal axis 112. For example, shaft 108 can define a curved profile. As shown, shaft 108 can include first shaft portion 108a and second shaft portion 108b; first shaft portion 108a extends distally from the base along the shaft longitudinal axis and second shaft portion 108b extends distally from the first shaft portion toward the tip. As shown, least a portion of second shaft portion 108b can be at an angle to the shaft longitudinal axis 112. One or more parts of shaft 108 can, in some instances, include facets, bevels, or other such features. One such feature is shown in FIG. 1A by facet 109. Without being bound to any particular theory or embodiment, such features can facilitate device usage, even device insertion.

Microneedle 100 can also include tip 110; as shown, the tip can be offset from shaft longitudinal axis 112. Microneedle 100 can include a lumen (not shown); the lumen can define a channel providing fluid communication within at least shaft 108 of microneedle 100. Microneedle 100 can also include opening 106, which opening can be defined at the distal end of the lumen and at a loci on shaft 108. Tip 114 can extend along tip longitudinal axis 114; as shown, tip longitudinal axis 114 can be at an angle (α) to shaft longitudinal axis 112. Angle α can be, for example, from greater than 0° to about 90°, for example from about 10° to about 80°, from about 15° to about 75°, from about 20° to about 70°, from about 25° to about 65°, from about 30° to about 60°, from about 35° to about 55°, from about 40° to about 50°, or even about 45°. As explained elsewhere herein, a microneedle according to the present disclosure can include a single lumen or a plurality of lumens.

Shaft 108 can have an outer diameter of, for example, from about 50 to about 250 μm, although this is not a requirement. The outer diameter can be, for example, from about 50 to about 250 μm, from about 60 to about 240 μm, from about 70 to about 230 μm, from about 80 to about 220 μm, from about 90 to about 210 μm, from about 100 to about 200 μm, from about 120 to about 180 μm, from about 135 to about 165 μm, and all intermediate ranges. Outer diameters of from about 50 to about 100 μm are considered particularly suitable, but are not required. A lumen can have a diameter of from about 30 to about 150 μm, for example from about 40 to about 140 μm, from about 50 to about 130 μm, from about 60 to about 120 μm, from about 70 to about 110 μm, from about 80 to about 100 μm, or even about 90 μm.

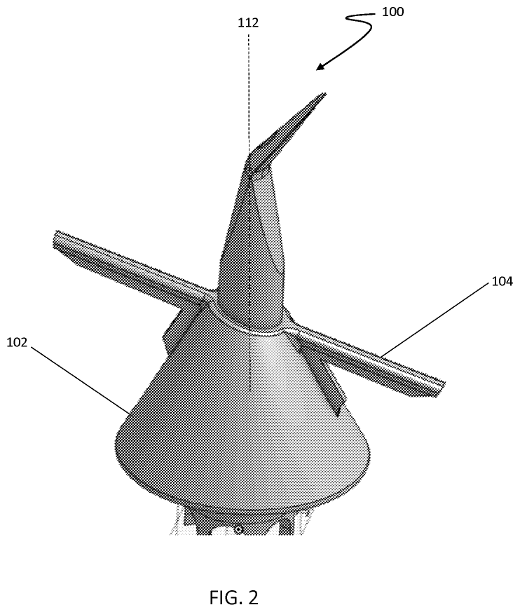

Microneedle 100 can also include one or more wings 104. A wing can be resiliently biased toward a configuration that extends outwardly from base 102. As but one example, wing 104 can bend or otherwise collapse so as to fit within a portal as shown in FIG. 1, and wing 104 can then unfold as shown in FIG. 2 when the wing is no longer constrained, for example when the microneedle is removed from the portal. As shown in FIG. 1, wing 104 (or another extension or marker of the microneedle) can lie in a plane or otherwise be parallel with tip 110 and/or with longitudinal axis 114, but this is not a requirement. Such an arrangement can assist a user in inserting tip 110 parallel to the vein into which the microneedle is inserted; aligning the microneedle such that the wing is parallel to the blood vessel will also align the microneedle tip parallel to the blood vessel, thereby allowing for insertion of the tip along the direction of the blood vessel.

Wing 104 can be bendable so that at least a portion of wing 104 extends generally upwardly or downwardly. Such a bending is shown in FIG. 1, in which wing 104 is bent to extend generally along shaft longitudinal axis 112. When the bending force is released from wing 104, the wing can return to a configuration in which wing 104 extends generally outwardly from base 102, as shown in FIG. 2. In some embodiments, wing 104 can be resilient and constructed such that as tip 114 is advanced deeper and deeper into a vessel, wing 104 bends, thereby providing the user with an indication of how deeply tip 114 has entered into the tissue of interest. The end 104a of a wing can be rounded, beveled, or otherwise shaped so as to facilitate movement of the end 104a of wing 104; this can, for example, be to facilitate sliding of the end 104a of wing 104 as microneedle 100 is advanced into a vessel, and the end 104a slides along the outer surface of the vessel as wing 104 bends and microneedle 100 is advanced. The resiliency of wing 104 can, in some instances, serve to oppose insertion of tip 114 into a tissue, thereby providing the user with additional control and/or feedback over tip insertion into a vessel.

As shown, wing 104 can be placed so as to contact a tissue into which tip 110 is inserted as microneedle 100 is advanced into the tissue. Without being bound to any particular theory or embodiment, wing 104 can serve as a visual indicator to a user that the wing has contacted tissue, for example when the wing is seen to flex. Wing 104 can also serve to stabilize microneedle 100 when microneedle 100 has been inserted, and wing 104 can even oppose or even prevent excessively deep insertion of microneedle 100 into a tissue.

Wing 104 or other extension from the microneedle can serve as a visual guide to the user to assist in aligning the microneedle along a blood vessel into which the microneedle is being inserted. As an example, a wing or other extension can extend from the microneedle in a direction that is parallel to the angled tip of the microneedle, thereby providing the user with a visual indicator of the direction in which the angled tip of the microneedle extends. It should be understood that a microneedle according to the present disclosure can, in addition to or in place of a wing or other extension, include a visible marker or other indicia used to guide alignment of the microneedle in use.

As shown, base 102 can be conical or even frustoconical in configuration. Base 102 can be configured to mount a medical instrument, the medical instrument optionally being a syringe. As but one example, a microneedle according to the present disclosure can be mounted on a cannula, which mounting can facilitate insertion of the microneedle into the eye or other structure. Base 102 can include, as examples, a skirt, tabs, prongs, and the like to facilitate attachment of the microneedle to a medical instrument.

FIG. 2 provides an alternative embodiment of the disclosed microneedles. By comparison to FIG. 1, wing 104 in FIG. 2 is shown extending generally outwardly from base 102.

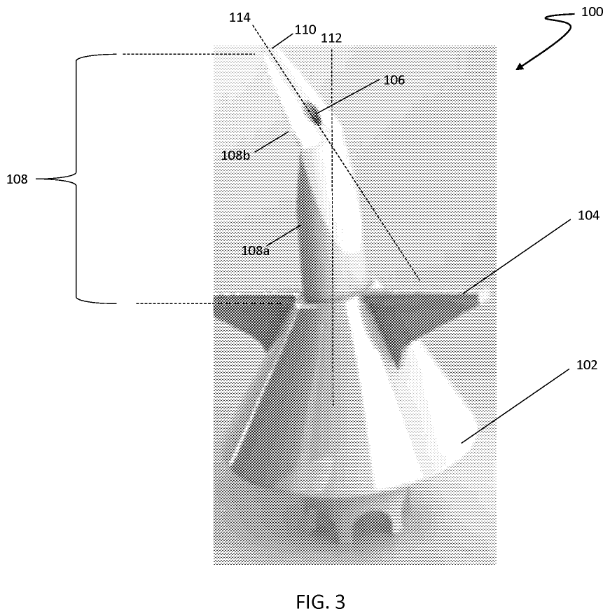

FIG. 3 provides an alternative embodiment of the disclosed microneedles. By comparison to FIG. 1, wing 104 in FIG. 3 is shown extending generally outwardly from base 102 and can be made from a comparatively rigid material.

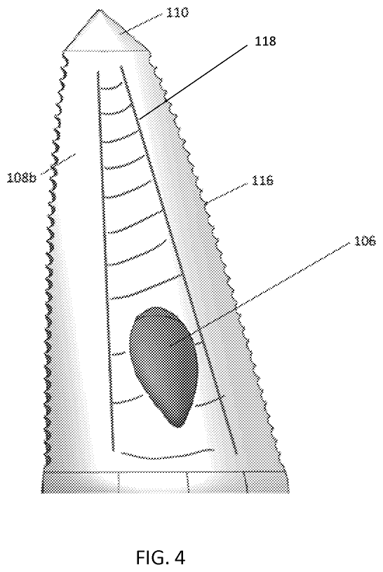

FIG. 4 provides a view of a second shaft portion 108 of a microneedle according to the present disclosure. As shown, second shaft portion can include opening 106 as well as serrated edge 116, which serrated edge 116 can extend toward and to tip 110. Without being bound to any particular theory or embodiment, serrated edge 116 can facilitate insertion of microneedle 100 into the tissue of a subject; the serrated edge creates smaller cuts into the tissue, reducing the insertion force required to cut and subsequent tissue damage. This in turn reduces complications, patient recovery time, and associated healthcare costs. Without being bound to any particular theory or embodiment, at least a portion of a microneedle as described herein can be manufactured using 2-photon lithography, which approach confers a high level of quality control. A serrated edge can have an individual sawtooth size of, for example, about 1.5 micrometers. This is not, however, a requirement.

As shown in FIG. 4, a microneedle according to the present disclosure can include an open channel 118 on a surface on which opening 106 is present. Without being bound to any particular theory or embodiment, open channel 118 can be useful during an aspiration process, as the presence of such a channel can prevent a wall of a blood vessel—such as a vein—from being sucked up against opening 106, thereby blocking the flow. The presence of channel 118 can mitigate such an effect.

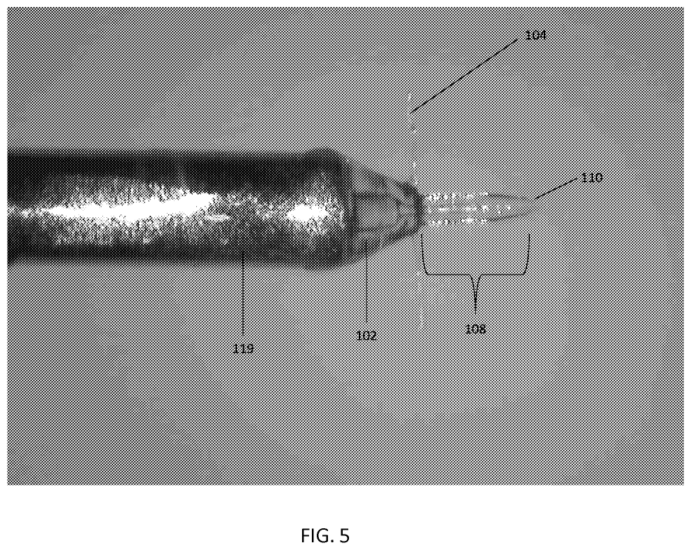

FIG. 5 provides an illustration of a microneedle according to the present disclosure mounted on a medical instrument 118. As shown, base 102 is engaged with medical instrument 119. Medical instrument 119 in FIG. 5 is a syringe needle, but the microneedles according to the present disclosure are not limited to mounting to syringes, as the disclosed microneedles can mount to other instruments, such as cannulas, and the like. As shown, shaft 108 extends from base 102 towards tip 110, and wings 104 extend generally outwardly (e.g., radially outwardly related to the shaft longitudinal axis, not shown in FIG. 5) from base 102.

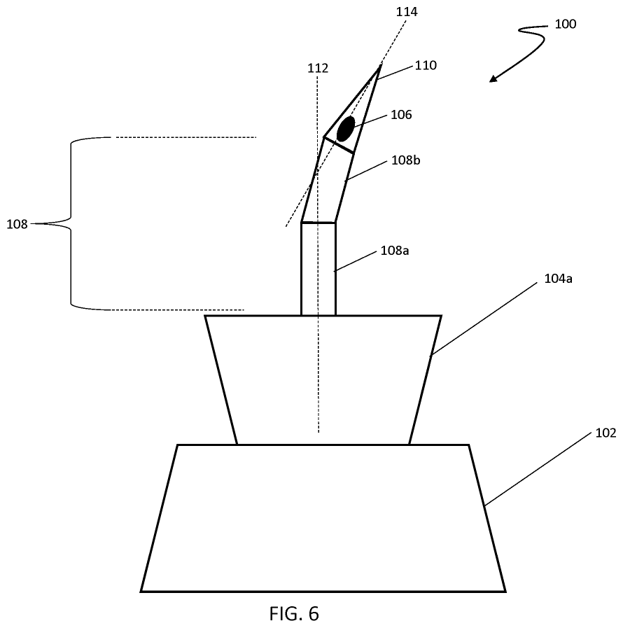

FIG. 6 provides a depiction of an exemplary microneedle 100 according to the present disclosure. By comparison to FIG. 1, microneedle 100 includes collar 104a, which can serve the same purpose as wing 104 in FIG. 1. Without being bound to any particular theory or embodiment, collar 104a can serve as a visual indicator to a user that the collar has contacted tissue, for example when the collar is seen to flex. Collar 104a can also serve to stabilize microneedle 100 when microneedle 100 has been inserted, and collar 104a can even oppose or even prevent excessively deep insertion of microneedle 100 into a tissue.

Collar 104a can include one or more markers, indicia, and/or extensions that can be used by the operator to align microneedle 100 relative to a vein or other structure into which the microneedle is inserted. Collar 104a can be axisymmetric in configuration, but this is not a requirement. Collar 104a can be opaque, but can also be translucent or even transparent. As shown, collar 104a can be conical in shape, and can be an inverted and/or truncated cone. Collar 104a can be circular in cross-section, but this is not a requirement, as collar 104a can be ovoid, polygonal, or otherwise noncircular in cross-section. Also as shown in FIG. 6, shaft 108 can define a profile that deviates from shaft longitudinal axis 112. For example, shaft 108 can define a curved profile. As shown, shaft 108 can include first shaft portion 108a and second shaft portion 108b; first shaft portion 108a extends distally from the base along the shaft longitudinal axis and second shaft portion 108b extends distally from the first shaft portion toward the tip. As shown, least a portion of second shaft portion 108b can be at an angle to the shaft longitudinal axis 112. Tip 114 can extend along tip longitudinal axis 114; as shown, tip longitudinal axis 114 can be at an angle to shaft longitudinal axis 112. As explained elsewhere herein, a microneedle according to the present disclosure can include a single lumen or a plurality of lumens.

### Example

In an example, two-photon polymerization lithography was employed to fabricate microneedles (80 μm outer diameter/60 μm inner diameter) with ultrasharp tips and curved lumina. These microneedles underwent a rigorous testing process, including several different designs tested for the ease of entering the retinal vessels using freshly enucleated (<12 hours) pig eyes. Design modifications were made to puncture the retinal vessels in a minimally damaging fashion and increase their cannulation ability, ensuring the utmost safety of the microneedle.

An 80 μm microneedle (60 μm inner diameter) with a curved tip was used to puncture the retinal vessels. This design—see FIG. 1A—allowed the surgeon to visualize the needle tip and correctly position it over the retinal vessels with utmost precision. A serrated blade-like manufacturing of the microneedle's edge allowed it to slice the upper vessel wall along the vessel wall fiber direction. Wing-like extensions were added to the design to be able to align the needle tip with the vessel that will be cannulated and limit the insertion depth of the needle, thus preventing the perforation of the retinal vessel wall. Using the finalized design, retinal vessels were entered and perfused without any difficulty, as shown in FIGS. 1A, 1B, and 1C.

The disclosed microneedles thus represent a significant advancement in the field and allow perfusion of the retinal vessels intraoperatively. This breakthrough technology has application to treatment of several retinal diseases, including retinal vascular occlusions, subretinal gene therapy, removal of subretinal perfluorocarbon droplets, and selective chemotherapy of retinal malignancies.

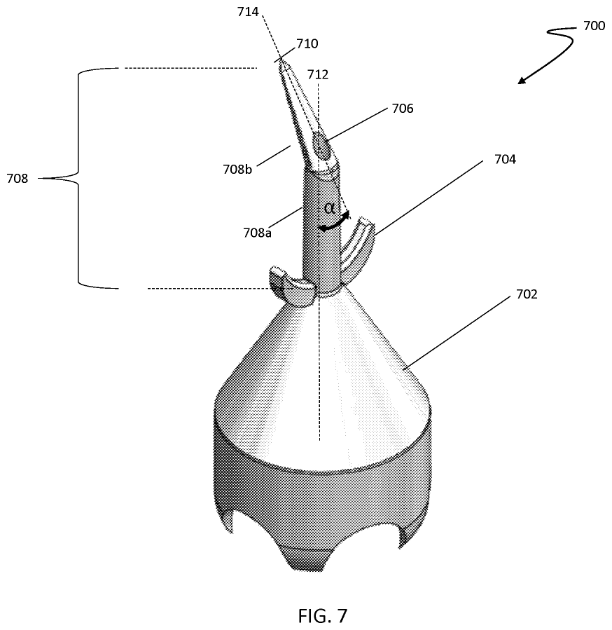

FIG. 7 provides a depiction of an exemplary microneedle 700 according to the present disclosure. As shown, microneedle 700 can include a base 702. Shaft 708 can extend distally from base 702; at least a portion of shaft 708 can extend along shaft longitudinal axis 712.

Shaft 708 can define a profile that deviates from shaft longitudinal axis 712. For example, shaft 708 can define a curved profile. As shown, shaft 708 can include first shaft portion 708a and second shaft portion 708b; first shaft portion 708a extends distally from the base along the shaft longitudinal axis and second shaft portion 708b extends distally from the first shaft portion toward the tip. As shown, least a portion of second shaft portion 708b can be at an angle to the shaft longitudinal axis 712. One or more parts of shaft 708 can, in some instances, include facets, bevels, or other such features. Without being bound to any particular theory or embodiment, such features can facilitate device usage, even device insertion.

Microneedle 700 can also include tip 710; as shown, the tip can be offset from shaft longitudinal axis 712. Microneedle 700 can include a lumen (not shown); the lumen can define a channel providing fluid communication within at least shaft 708 of microneedle 700. Microneedle 700 can also include opening 706, which opening can be defined at the distal end of the lumen and at a loci on shaft 708. Tip 710 can extend along tip longitudinal axis 714; as shown, tip longitudinal axis 714 can be at an angle (α) to shaft longitudinal axis 712. Angle α can be, for example, from greater than 0° to about 90°, for example from about 10° to about 80°, from about 15° to about 75°, from about 20° to about 70°, from about 25° to about 65°, from about 30° to about 60°, from about 35° to about 55°, from about 40° to about 50°, or even about 45°. As explained elsewhere herein, a microneedle according to the present disclosure can include a single lumen or a plurality of lumens.

Shaft 708 can have an outer diameter of, for example, from about 30 to about 45 μm, although this is not a requirement. The outer diameter can be, for example, from about 30 to about 250 μm, from about 30 to about 240 μm, from about 30 to about 230 μm, from about 30 to about 220 μm, from about 30 to about 210 μm, from about 30 to about 200 μm, from about 30 to about 180 μm, from about 30 to about 165 μm, and all intermediate ranges. Outer diameters of from about 30 to about 45 μm are considered particularly suitable, but are not required.

Microneedle 700 can also include one or more wings 704. A wing can be resiliently biased toward a configuration that extends outwardly from base 702. As but one example, wing 704 can bend or otherwise collapse so as to fit within a portal, and wing 704 can then unfold when the wing is no longer constrained, for example when the microneedle is removed from the portal. As shown in FIG. 7, wing 704 (or another extension or marker of the microneedle) can lie in a plane or otherwise be parallel with tip 710 and/or with tip longitudinal axis 714, but this is not a requirement. Wing 704 can extend perpendicular relative to tip longitudinal axis 714. As but one example, wing 704 can extend generally perpendicular to a plane in which shaft longitudinal axis 712 and tip longitudinal axis 714 lie. Wing 704 can define an arc shape; a non-limiting example of such a shape is shown in FIG. 7. Wing 704 can define, for example, a curvature of a constant radius, but this is not a requirement, as wing 704 can include a curvature that is not of a constant radius. Without being bound to any particular theory or embodiment, an arc-shaped wing can lie along the surface of a blood vessel into which the microneedle is inserted, thereby imparting stability for injection. Wing 704 can also be used to limit the depth of insertion of the microneedle into a vessel, thereby preventing overly-deep insertions of the needles.

Such an arrangement can assist a user in inserting tip 710 parallel to the vein into which the microneedle is inserted; aligning the microneedle such that the wing is parallel to the blood vessel will also align the microneedle tip parallel to the blood vessel, thereby allowing for insertion of the tip along the direction of the blood vessel.

Wing 704 can be bendable so that at least a portion of wing 704 extends generally upwardly or downwardly. Such a bending is shown in FIG. 7, in which wing 704 is bent to extend generally along shaft longitudinal axis 712. When the bending force is released from wing 704, the wing can return to a configuration in which wing 704 extends generally outwardly from base 702. In some embodiments, wing 704 can be resilient and constructed such that as tip 714 is advanced deeper and deeper into a vessel, wing 704 bends, thereby providing the user with an indication of how deeply tip 714 has entered into the tissue of interest. The end 704a of a wing can be rounded, beveled, or otherwise shaped so as to facilitate movement of the end 704a of wing 704; this can, for example, be to facilitate sliding of the end 704a of wing 704 as microneedle 700 is advanced into a vessel, and the end 704a slides along the outer surface of the vessel as wing 704 bends and microneedle 700 is advanced. The resiliency of wing 704 can, in some instances, serve to oppose insertion of tip 714 into a tissue, thereby providing the user with additional control and/or feedback over tip insertion into a vessel.

As shown, wing 704 can be placed so as to contact a tissue into which tip 710 is inserted as microneedle 700 is advanced into the tissue. Without being bound to any particular theory or embodiment, wing 704 can serve as a visual indicator to a user that the wing has contacted tissue, for example when the wing is seen to flex. Wing 704 can also serve to stabilize microneedle 700 when microneedle 700 has been inserted, and wing 704 can even oppose or even prevent excessively deep insertion of microneedle 700 into a tissue.

Wing 704 or other extension from the microneedle can serve as a visual guide to the user to assist in aligning the microneedle along a blood vessel into which the microneedle is being inserted. As an example, a wing or other extension can extend from the microneedle in a direction that is parallel to the angled tip of the microneedle, thereby providing the user with a visual indicator of the direction in which the angled tip of the microneedle extends. It should be understood that a microneedle according to the present disclosure can, in addition to or in place of a wing or other extension, include a visible marker or other indicia used to guide alignment of the microneedle in use.

As shown, base 702 can be conical or even frustoconical in configuration. Base 702 can be configured to mount a medical instrument, the medical instrument optionally being a syringe. As but one example, a microneedle according to the present disclosure can be mounted on a cannula, which mounting can facilitate insertion of the microneedle into the eye or other structure. Base 702 can include, as examples, a skirt, tabs, prongs, and the like to facilitate attachment of the microneedle to a medical instrument.

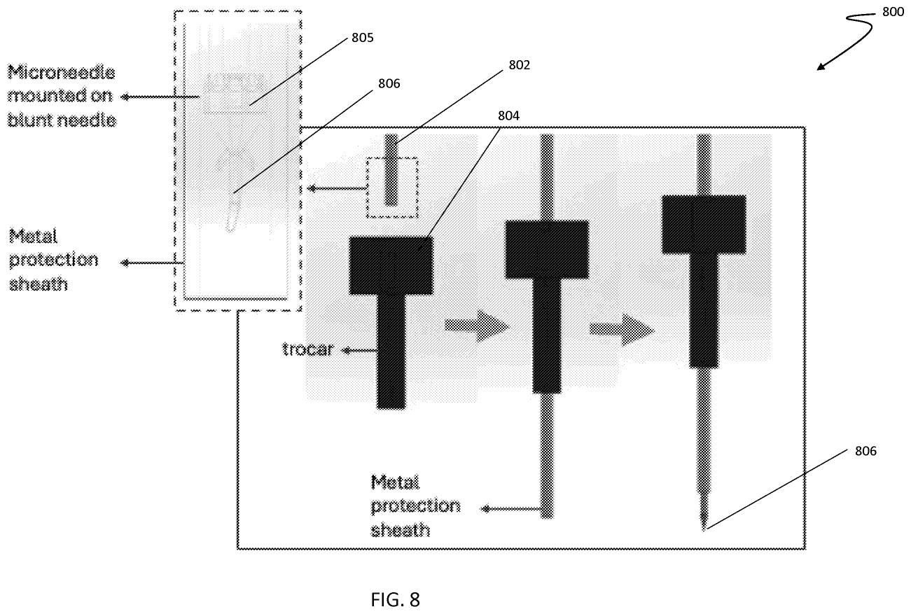

FIG. 8 provides a schematic of the microneedle 806 mounted on a blunt needle 805 and enclosed within a protective sheath 802 for safe insertion through the trocar. As shown, a microneedle according to the present disclosure can be mounted on a larger or “blunt” needle. The microneedle-needle assembly can then be enclosed within protective sheath 802. The protective sheath can then be inserted into and then through trocar 804, as shown in FIG. 8. After insertion through the trocar, the microneedle can be extended beyond the end of protective sheath 802, and the user can then use the microneedle for injection, aspiration, or other actions.

### ASPECTS

The following Aspects are illustrative only and do not limit the scope of the present disclosure or the appended claims. Any part or parts of any one or more Aspects can be combined with any part or parts of any one or more other Aspects.

### Aspect 1

A microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis; and a plurality of wings extending outwardly from the base, the plurality of wings being configured to contact a tissue into which the tip is inserted.

A lumen can, for example, extend through the base to the shaft and then on to the opening of the microneedle.

### Aspect 2

The microneedle of Aspect 1, wherein the base is configured to mount a medical instrument, the medical instrument optionally being a syringe.

### Aspect 3

The microneedle of any one of Aspects 1-2, wherein the shaft defines a profile that deviates from the shaft longitudinal axis.

### Aspect 4

The microneedle of Aspect 3, wherein the shaft defines a curved profile.

### Aspect 5

The microneedle of Aspect 3, wherein the shaft comprises (1) a first shaft portion that extends distally from the base along the shaft longitudinal axis and (2) a second shaft portion that extends distally from the first shaft portion toward the tip, at least a portion of the second shaft portion being at an angle to the shaft longitudinal axis.

### Aspect 6

The microneedle of any one of Aspects 1-5, wherein a wing is configured as being resiliently biased toward a configuration that extends outwardly from the base.

### Aspect 7

The microneedle of any one of Aspects 1-6, wherein the shaft defines at least one edge extending distally toward the tip.

### Aspect 8

The microneedle of Aspect 7, the at least one edge being serrated.

### Aspect 9

The microneedle of any one of Aspects 1-8, wherein the at least one lumen is a single lumen.

### Aspect 10

The microneedle of any one of Aspects 1-9, wherein the at least one lumen is two lumens, optionally one of the two lumens being configured to inject a fluid and the other of the two lumens being configured to aspirate a fluid.

### Aspect 11

The microneedle of any one of Aspects 1-10, wherein the microneedle is dimensioned so as to fit within an ophthalmic portal.

### Aspect 12

The microneedle of Aspect 11, wherein the plurality of wings are collapsible such that the microneedle fits within an ophthalmic portal.

### Aspect 13

A microneedle, comprising: a base disposed at a proximal end of the microneedle; a tip disposed at a distal end of the microneedle, the tip extending along a tip longitudinal axis; a shaft disposed between the base and the tip, the shaft extending distally from the base along a shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis; and at least one extension extending from the base, the at least one extension configured to contact a tissue into which the tip is inserted, the at least one extension comprising at least one of a wing and a collar.

### Aspect 14

The microneedle of Aspect 13, wherein an extension is configured as being resiliently biased toward a configuration that extends from the base.

### Aspect 15

The microneedle of any one of Aspects 13-14, wherein the shaft defines at least one edge extending distally toward and to the tip, the at least one edge optionally being serrated.

### Aspect 16

The microneedle of any one of Aspects 13-15, wherein the shaft defines a profile that deviates from the shaft longitudinal axis, the shaft optionally defining at least one of a curved profile and an angled profile.

### Aspect 17

The microneedle of any one of Aspects 13-16, wherein the microneedle is dimensioned so as to fit within an ophthalmic portal.

### Aspect 18

A microneedle, comprising: a base disposed at a proximal end of the microneedle; a tip disposed at a distal end of the microneedle, the tip extending along a tip longitudinal axis, a shaft disposed between the base and the tip, wherein the shaft defines at least one edge extending distally toward and to the tip, the at least one edge being serrated; optionally, at least one lumen defining a channel providing fluid communication within at least the shaft of the microneedle; and optionally, an opening defined at the distal end of the lumen and at a loci on the shaft, wherein the opening is optionally offset from the tip longitudinal axis.

### Aspect 19

A microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, wherein the shaft defines a profile that deviates from the shaft longitudinal axis, the shaft optionally defining at least one of a curved profile and an angled profile, and the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis.

### Aspect 20

A microneedle, comprising: a base disposed at a proximal end of the microneedle; a tip disposed at a distal end of the microneedle, the tip extending along a tip longitudinal axis, the base having a width greater than the width of the tip; a shaft disposed between the base and the tip, the shaft extending distally from the base along a shaft longitudinal axis, the tip optionally being offset from the shaft longitudinal axis; at least one lumen defining a channel providing fluid communication within at least the shaft of the microneedle; an opening defined at the distal end of the lumen and at a loci on the shaft, wherein the opening is optionally offset from the tip longitudinal axis; and a plurality of wings extending outwardly from the base, the plurality of wings being configured to contact a tissue into which the tip is inserted, at least one of the plurality of wings lying perpendicular to a plane in which the shaft longitudinal axis and the tip longitudinal axis lie.

### Aspect 21

The microneedle of Aspect 20, wherein the base is configured to mount a medical instrument, the medical instrument optionally being a syringe.

### Aspect 22

The microneedle of any one of Aspects 20-21, wherein the shaft defines a profile that deviates from the shaft longitudinal axis.

### Aspect 23

The microneedle of Aspect 22, wherein the shaft defines a curved profile.

### Aspect 24

The microneedle of Aspect 22, wherein the shaft comprises (1) a first shaft portion that extends distally from the base along the shaft longitudinal axis and (2) a second shaft portion that extends distally from the first shaft portion toward the tip, at least a portion of the second shaft portion being at an angle to the shaft longitudinal axis.

### Aspect 25

The microneedle of any one of Aspects 20-24, wherein a wing is configured as being resiliently biased toward a configuration that extends outwardly from the base.

### Aspect 26

The microneedle of any one of Aspects 20-25, wherein the shaft defines at least one edge extending distally toward the tip.

### Aspect 27

The microneedle of Aspect 26, the at least one edge being serrated.

### Aspect 28

The microneedle of any one of Aspects 20-27, wherein the at least one lumen is a single lumen.

### Aspect 29

The microneedle of any one of Aspects 20-27, wherein the at least one lumen is two lumens, optionally one of the two lumens being configured to inject a fluid and the other of the two lumens being configured to aspirate a fluid.

### Aspect 30

The microneedle of any one of Aspects 20-29, wherein the microneedle is dimensioned so as to fit within an ophthalmic portal and optionally wherein the plurality of wings are collapsible such that the microneedle fits within the ophthalmic portal.

### Aspect 31

The microneedle of any one of Aspects 20-30, wherein a wing defines an arc shape, the arc shape optionally having a curvature of a constant radius.

### Aspect 32

The microneedle of any one of Aspects 20-31, wherein the shaft has a diameter of from about 30 to about 50 micrometers.

### Aspect 33

A method, comprising effecting injection or aspiration of an intraocular blood vessel with a microneedle according to any one of Aspects 20-32.

### Aspect 34

A method, comprising delivering a fluid to a subject or withdrawing a fluid from a subject using one or more microneedles according to any one of Aspects 1-32. A fluid can be, for example, a buffer, a therapeutic, a dye, and the like. A fluid can comprise a protein, a small molecule, a nanoparticle, and the like. The microneedles according to the present disclosure can be used for either or both of treatment purposes or diagnostic purposes.

In treating a patient, a user can use a single microneedle according to the present disclosure. A user can, however, also use a plurality of microneedles according to the present disclosure. As an example one can use two (or more) two microneedles, with each microneedle including one or more lumens. The use of two (or more) microneedles can be effected simultaneously when treating a patient. As but one example, one can use a first microneedle according to the present disclosure to perforate into a vein at a location that is “downstream” of an occlusion of the vein and use a second microneedle according to the present disclosure to perforate into the vein at a location “upstream” of an occlusion. In this way, one can use the first microneedle to introduce a therapeutic to the vein to treat the occlusion, and one can use the second microneedle to aspirate material downstream of the occlusion. One can also use a single microneedle to aspirate and inject material.

What is claimed:

1. A microneedle, comprising:
a base disposed at a proximal end of the microneedle;
a tip disposed at a distal end of the microneedle,
the tip extending along a tip longitudinal axis,
the base having a width greater than the width of the tip;
a shaft disposed between the base and the tip,
the shaft extending distally from the base along a shaft longitudinal axis,
the tip optionally being offset from the shaft longitudinal axis;
at least one lumen defining a channel providing fluid communication within at least the shaft of the microneedle;
an opening defined at the distal end of the lumen and at a loci on the shaft,
wherein the opening is optionally offset from the tip longitudinal axis; and
a plurality of wings extending outwardly from the base,
the plurality of wings being configured to contact a tissue into which the tip is inserted.

2. The microneedle of claim 1, wherein the base is configured to mount a medical instrument, the medical instrument optionally being a syringe.

3. The microneedle of claim 1, wherein the shaft defines a profile that deviates from the shaft longitudinal axis.

4. The microneedle of claim 3, wherein the shaft defines a curved profile.

5. The microneedle of claim 3, wherein the shaft comprises (1) a first shaft portion that extends distally from the base along the shaft longitudinal axis and (2) a second shaft portion that extends distally from the first shaft portion toward the tip, at least a portion of the second shaft portion being at an angle to the shaft longitudinal axis.

6. The microneedle of claim 1, wherein a wing is configured as being resiliently biased toward a configuration that extends outwardly from the base.

7. The microneedle of claim 1, wherein the shaft defines at least one edge extending distally toward the tip.

8. The microneedle of claim 7, the at least one edge being serrated.

9. The microneedle of claim 1, wherein the at least one lumen is a single lumen.

10. The microneedle of claim 1, wherein the at least one lumen is two lumens, optionally one of the two lumens being configured to inject a fluid and the other of the two lumens being configured to aspirate a fluid.

11. The microneedle of claim 1, wherein the microneedle is dimensioned so as to fit within an ophthalmic portal.

12. The microneedle of claim 11, wherein the plurality of wings are collapsible such that the microneedle fits within the ophthalmic portal.

13. A microneedle, comprising:
a base disposed at a proximal end of the microneedle;
a tip disposed at a distal end of the microneedle,
the tip extending along a tip longitudinal axis;
a shaft disposed between the base and the tip,
the shaft extending distally from the base along a shaft longitudinal axis;
at least one lumen defining a channel providing fluid communication within at least the shaft of the microneedle;
an opening defined at the distal end of the lumen and at a loci on the shaft,
wherein the opening is optionally offset from the tip longitudinal axis; and
at least one extension extending from the base,
the at least one extension configured to contact a tissue into which the tip is inserted, the at least one extension comprising at least one of a wing and a collar.

14. The microneedle of claim 13, wherein an extension is configured as being resiliently biased toward a configuration that extends from the base.

15. The microneedle of claim 13, wherein the shaft defines at least one edge extending distally toward and to the tip, the at least one edge optionally being serrated.

16. The microneedle of claim 13, wherein the shaft defines a profile that deviates from the shaft longitudinal axis, the shaft optionally defining at least one of a curved profile and an angled profile.

17. The microneedle of claim 13, wherein the microneedle is dimensioned so as to fit within an ophthalmic portal.

18. A microneedle, comprising:
a base disposed at a proximal end of the microneedle;
a tip disposed at a distal end of the microneedle,
the tip extending along a tip longitudinal axis,
a shaft disposed between the base and the tip,
wherein the shaft defines at least one edge extending distally toward and to the tip, the at least one edge being serrated;
optionally, at least one lumen defining a channel providing fluid communication within at least the shaft of the microneedle; and
optionally, an opening defined at the distal end of the lumen and at a loci on the shaft,
wherein the opening is optionally offset from the tip longitudinal axis.

19. A microneedle, comprising:
a base disposed at a proximal end of the microneedle;
a tip disposed at a distal end of the microneedle,
the tip extending along a tip longitudinal axis,
the base having a width greater than the width of the tip;
a shaft disposed between the base and the tip,
the shaft extending distally from the base along a shaft longitudinal axis,
wherein the shaft defines a profile that deviates from the shaft longitudinal axis, the shaft optionally defining at least one of a curved profile and an angled profile, and
the tip optionally being offset from the shaft longitudinal axis;
at least one lumen defining a channel providing fluid communication within at least the shaft of the microneedle;
an opening defined at the distal end of the lumen and at a loci on the shaft,
wherein the opening is optionally offset from the tip longitudinal axis.

20. A microneedle, comprising:
a base disposed at a proximal end of the microneedle;
a tip disposed at a distal end of the microneedle,
the tip extending along a tip longitudinal axis, the base having a width greater than the width of the tip;
a shaft disposed between the base and the tip, the shaft extending distally from the base along a shaft longitudinal axis,
the tip optionally being offset from the shaft longitudinal axis;
at least one lumen defining a channel providing fluid communication within at least the shaft of the microneedle; an opening defined at the distal end of the lumen and at a loci on the shaft,
wherein the opening is optionally offset from the tip longitudinal axis; and
a plurality of wings extending outwardly from the base,
the plurality of wings being configured to contact a tissue into which the tip is inserted, at least one of the plurality of wings lying perpendicular to a plane in which the shaft longitudinal axis and the tip longitudinal axis lie.
